(19) United States
(12) Patent Application Publication
(10) Pub. No.: US 2021/0283107 A1
(43) Pub. Date: Sep. 16, 2021

(54) Methods to Expand the Spectrum of Gram-Positive Antibiotics

(71) Applicant: NORTH CAROLINA STATE UNIVERSITY, Raleigh, NC (US)

(72) Inventors: Christian Melander, Raleigh, NC (US); Roberta Melander, Raleigh, NC (US)

(21) Appl. No.: 16/325,602

(22) PCT Filed: Aug. 14, 2017
(86) PCT No.: PCT/US2017/046712

§ 371 Date: Feb. 14, 2019

Related U.S. Application Data
(60) Provisional application No. 62/375,077, filed on Aug. 15, 2016.

(51) Int. Cl. A61K 31/4168; A61K 31/7048; A61K 31/7052; A61P 31/04; A61K 38/14; A61K 38/12; A61K 31/5377

(52) U.S. Cl. CPC A61K 31/4168; A61K 31/7048; A61K 31/7052; A61K 31/5377; A61K 38/14; A61K 38/12; A61P 31/04

(57) ABSTRACT

Provided herein is a method to treat a Gram-negative bacterial infection in a subject in need thereof, comprising administering a compound of Formula I: in combination with a Gram-positive antibiotic to said subject in a treatment-effective amount. Also provided is a method to control Gram-negative bacteria, comprising applying a compound of Formula I in combination with a Gram-positive antibiotic in a Gram-negative bactericidal-effective amount.

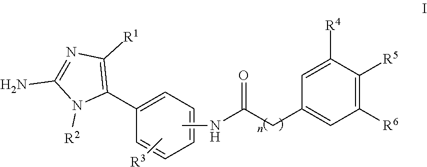

### STATEMENT OF GOVERNMENT SUPPORT

This invention was made with Government support under grant number GM055769 awarded by the National Institutes of Health. The Government has certain rights to this invention.

### BACKGROUND

The increasing prevalence of multi drug resistant (MDR) bacteria is a serious national and global threat. As an alternative to developing new antibiotics, our group and others have developed adjuvant molecules that potentiate the activity of antibiotics to which the bacteria are resistant. Many examples of the use of antibiotic adjuvants involve suppressing acquired mechanisms of resistance, such as ß-lactamase mediated resistance to ß-lactam antibiotics and phosphoethanolamine transferase mediated resistance to polymyxins. Brackett et al., J. Med. Chem. 2014, 57, 7450-7458, describes 2-aminoimidazole compounds that can suppress acquired beta-lactam antibiotic drug resistance in Gram-negative pathogens.

In addition to acquired resistance, many species of bacteria are intrinsically resistant to certain classes of antibiotics. See Brown, E. D. and G. D. Wright, Antibacterial drug discovery in the resistance era. Nature, 2016. 529(7586): p. 336-43. This is particularly true in the case of Gram-negative bacteria, making the identification of antibiotics active against these bacteria significantly more difficult than those that are active against Gram-positive bacteria. Indeed, the last novel class of Gram-negative acting antibiotics to be put into the clinic was the fluoroquinolone class in the 1960s (Lewis, K., Platforms for antibiotic discovery. Nat Rev Drug Discov, 2013. 12(5): p. 371-87), and the current pipeline of antibiotics in development that are active against Gram-negative bacteria remains worryingly dry.

Gram staining is a traditional method of classifying bacteria based upon the staining differences of their cell walls. Gram-positive bacteria stain with a crystal violet dye and look purple or blue under a microscope, while Gram-negative bacteria do not retain the crystal violet dye but stain red or pink with a counterstain added after the crystal violet dye. The outer cell membrane of Gram-negative bacteria, which is lacking in Gram-positive bacteria, helps protect them from many of the antibiotics that can be used against Gram-positive bacteria, such as macrolides and glycopeptides.

There are several classes of clinically available antibiotics including macrolides, glycopeptides, lipopeptides and oxazolidinones that are active only against Gram-positive bacteria. Resistance to these antibiotic classes in Gram-negative bacteria is not due to the absence of the antibiotic target, but predominantly due to the inability of the antibiotic to access the target as a result of the impermeable nature of the Gram-negative outer membrane (in the case of the first three classes), and efflux of the antibiotic from the cell (in the case of oxazolidinones). Nikaido, H., Molecular basis of bacterial outer membrane permeability revisited. Microbiol Mol Biol Rev, 2003. 67(4): p. 593-656; Mollmann, U., et al., Siderophores as drug delivery agents: application of the “Trojan Horse” strategy. Biometals, 2009. 22(4): p. 615-24; Lambert, P. A., Cellular impermeability and uptake of biocides and antibiotics in gram-positive bacteria and mycobacteria. Symp Ser Soc Appl Microbiol, 2002(31): p. 46S-54S; Livermore, D. M., Antibiotic uptake and transport by bacteria. Scand J Infect Dis Suppl, 1990. 74: p. 15-22; Li, X. Z., P. Plesiat, and H. Nikaido, The challenge of efflux-mediated antibiotic resistance in Gram-negative bacteria. Clin Microbiol Rev, 2015. 28(2): p. 337-418; Leclercq, R., Mechanisms of Resistance to Macrolides and Lincosamides: Nature of the Resistance Elements and Their Clinical Implications Clinical Infectious Diseases, 2002. 34: p. 482-492.

These intrinsic resistance mechanisms represent an attractive adjuvant target, as overcoming them would expand the spectrum of both currently available Gram-positive selective antibiotics such as those mentioned above and also the likely higher numbers of Gram-positive acting antibiotics identified in future discovery efforts, and significantly increase the options available for treating some of the most serious bacterial infections. There have been some examples of the potentiation of Gram-positive selective antibiotics against Gram-negative bacteria, typically reliant on disruption of the outer membrane. See Viljanen, P. and M. Vaara, Susceptibility of gram-negative bacteria to polymyxin B nonapeptide. Antimicrob Agents Chemother, 1984. 25(6): p. 701-5; Ofek, I., et al., Antibacterial synergism of polymyxin B nonapeptide and hydrophobic antibiotics in experimental gram-negative infections in mice. Antimicrob Agents Chemother, 1994. 38(2): p. 374-7; Zabawa, T. P., et al., Treatment of Gram-negative bacterial infections by potentiation of antibiotics. Curr Opin Microbiol, 2016. 33: p. 7-12; Stokes, J. M., et al., Pentamidine sensitizes Gram-negative pathogens to antibiotics and overcomes acquired colistin resistance. Nat Microbiol, 2017. 2: p. 17028. However, the use of adjuvants to sensitize Gram-negative bacteria to Gram-positive selective antibiotics has been minimally explored.

U.S. 2009/0270475 to Melander et al. describes 2-aminoimidazole compounds that control biofilm formation. In some embodiments, the compounds may be administered in combination with a biocide such as an antibiotic.

Rogers et al., Antimicrobial Agents and Chemotherapy, May 2010, 2112-2118, describes the ability of a 2-aminoimidazole compound to resensitize resistant strains of Gram-positive (MRSA, S. aureus) and Gram-negative (E. coli, MDRAB) bacteria to their respective antibiotics.

Despite these advances, there remains a need to enhance the ability of antibiotics to combat a variety of bacterial infections.

### SUMMARY

Provided herein is a method to treat a Gram-negative bacterial infection in a subject in need thereof, comprising administering a compound of Formula I:

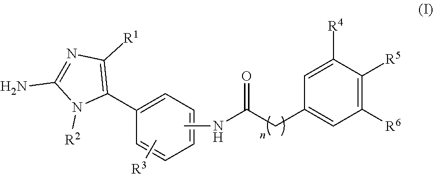

wherein:
R¹, R² and R³ are each independently H or alkyl (e.g., lower alkyl);
n is an integer from 0 to 5; and
R⁴, R⁵ and R⁶ are each independently H or halo,
or a pharmaceutically acceptable salt thereof,
in combination with a Gram-positive antibiotic to said subject in a treatment-effective amount.

In some embodiments, R⁵ is H; and R⁴ and R⁶ are each independently chloro or bromo.

In some embodiments, the compound of Formula I is a compound of Formula I(a):

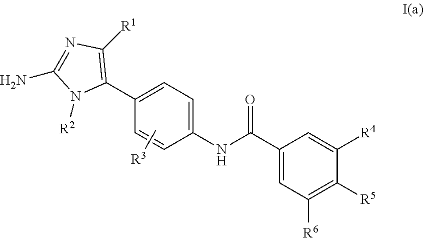

wherein:

R¹, R² and R³ are each independently H or alkyl (e.g., lower alkyl); and

R⁴, R⁵ and R⁶ are each independently H or halo,

or a pharmaceutically acceptable salt thereof.

In some embodiments, the compound of Formula I is:

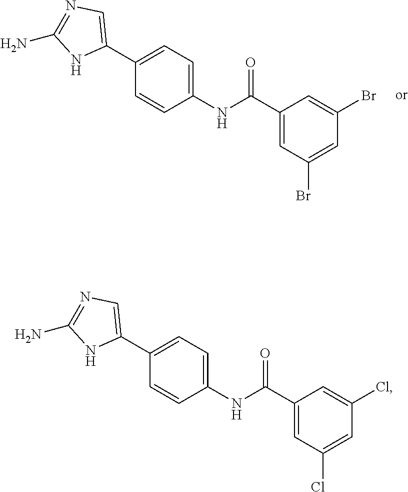 [SMILES: Nc1ncc(-c2ccc(NC(=O)c3cc(Br)cc(Br)c3)cc2)[nH]1 ; "or" ; Nc1ncc(-c2ccc(NC(=O)c3cc(Cl)cc(Cl)c3)cc2)[nH]1]

or a pharmaceutically acceptable salt thereof.

In some embodiments, the Cram-positive antibiotic is a macrolide antibiotic, a lipopeptide antibiotic, an oxazolidinone antibiotic, or a glycopeptide antibiotic.

In some embodiments, the Gram-negative bacterial infection comprises bacteria of the genus Acinetobacter, Escherichia, Salmonella, Vibrio, Klebsiella, and/or Helicobacter, e.g., Pseudomonas aeuroginosa, Bordetella pertussis, Vibrio vulnificus, Haemophilus influenzae, Halomonas pacifica, Klebsiella pneumoniae, and/or Acinetobacter baumannii.

Also provided is a method to control Gram-negative bacteria, comprising applying a compound of Formula I:

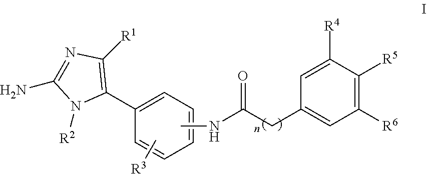

wherein:

R¹, R² and R³ are each independently H or alkyl (e.g., lower alkyl);

n is an integer from 0 to 5; and

R⁴, R⁵ and R⁶ are each independently H or halo,

or a pharmaceutically acceptable salt thereof,

in combination with a Gram-positive antibiotic in a Gram-negative bactericidal-effective amount.

In some embodiments, R⁵ is H; and R⁴ and R⁶ are each independently chloro or bromo.

In some embodiments, the compound of Formula I is a compound of Formula I(a):

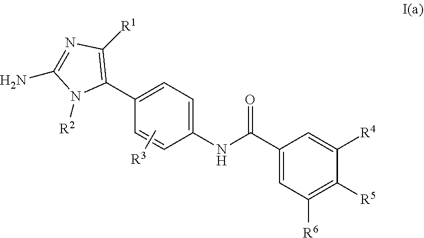

wherein:

R¹, R² and R³ are each independently H or alkyl (e.g., lower alkyl); and

R⁴, R⁵ and R⁶ are each independently H or halo,

or a pharmaceutically acceptable salt thereof.

In some embodiments, the compound of Formula I is:

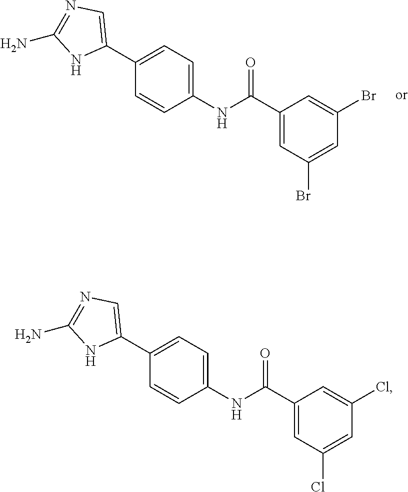 [SMILES: Nc1ncc(-c2ccc(NC(=O)c3cc(Br)cc(Br)c3)cc2)[nH]1 ; "or" ; Nc1ncc(-c2ccc(NC(=O)c3cc(Cl)cc(Cl)c3)cc2)[nH]1]

or a pharmaceutically acceptable salt thereof.

In some embodiments, the applying is carried out by applying the compound and Gram-positive antibiotic to a surface comprising or at risk of comprising said Gram-negative bacteria.

In some embodiments, the applying is carried out by adding the compound and Gram-positive antibiotic to a liquid or material (e.g., gels, foams, fabrics, etc.) comprising or at risk of comprising said Gram-negative bacteria.

In some embodiments, the Gram-positive antibiotic is a macrolide antibiotic, a lipopeptide antibiotic, an oxazolidinone antibiotic, or a glycopeptide antibiotic.

In some embodiments, the Gram-negative bacteria comprises bacteria of the genus Acinetobacter, Escherichia, Salmonella, Vibrio, Klebsiella, and/or Helicobacter, e.g., Pseudomonas aeuroginosa, Bordetella pertussis, Vibrio vulnificus, Naemophilus influenzae, Halomonas pacifica, Klebsiella pneumoniae, and/or Acinetobacter baumannii.

Also provided is the use of a compound as taught herein in the preparation of a medicament for treating a Gram-negative bacterial infection with a Gram-positive antibiotic. Further provided is the use of a compound taught herein for treating a Gram-negative bacterial infection with a Gram-positive antibiotic.

### BRIEF DESCRIPTION OF THE DRAWINGS

FIG. 1 shows a Kaplan Meier curve showing treatment of A. baumannii infection in G. mellonella model using combination therapy of compound 1 and clarithromycin. From bottom to top: A. baumannii only injection (“A baumannii”); Clarithromycin injection at 25 mg/kg (“CLARITHROMYCIN”); compound 1 injection at 100 mg/kg (“COMPOUND 1”); Clarithromycin and compound 1 combination injection (“CLARITHROMYCIN+COMPOUND 1”); Rifampin injection 30 mg/kg (“RIFAMPIN”).

### DETAILED DESCRIPTION OF EMBODIMENTS

The present invention is further described below. All patent references referred to in this patent application are hereby incorporated by reference in their entireties as if set forth fully herein.

As used herein in the description of the invention and the appended claims, the singular forms “a,” “an” and “the” are intended to include the plural forms as well, unless the context clearly indicates otherwise. Furthermore, the terms “about” and “approximately” as used herein when referring to a measurable value such as an amount of a compound, dose, time, temperature, and the like, is meant to encompass variations of 20%, 10%, 5%, 1%, 0.5%, or even 0.1% of the specified amount. Also, as used herein, “and/or” and “/” refer to and encompass any and all possible combinations of one or more of the associated listed items, as well as the lack of combinations when interpreted in the alternative (“or”).

### A. Definitions

“Imidazole” refers to the commonly-known structure:

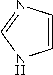

An “amino” is the group —NH₂.

As known in the art, “H” refers to a hydrogen atom. “C” refers to a carbon atom. “N” refers to a nitrogen atom. “O” refers to an oxygen atom.

“Halo” refers to F, Cl, Br or I. “Cl” is chloro, “I” is iodo, “F” is fluoro, and “Br” is bromo.

“Alkyl,” as used herein, refers to a straight or branched chain hydrocarbon containing from 1 or 2 to 10 or 20 or more carbon atoms (e.g., C2, C3, C4, C5, C6, C7, C8, C9, C10, C11, C12, C13, C14, C15, etc.). Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like. In some embodiments, the alkyl is a “lower alkyl” having from 1 to 3, 4, or 5 carbon atoms.

A “pharmaceutically acceptable salt” is a salt that retains the biological effectiveness of the free acids and bases of a specified compound and that is not biologically or otherwise undesirable. Examples of pharmaceutically acceptable salts include sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycollates, tartrates, methane-sulfonates, propanesulfonates, naphthalene-1-sulfonates, naphthalene-2-sulfonates, and mandelates.

### B. Active Compounds

Active compounds as described herein can be prepared as detailed elsewhere, e.g., in U.S. 2009/0270475 to Melander et al., or in accordance with known procedures or variations thereof that will be apparent to those skilled in the art.

As will be appreciated by those of skill in the art, the active compounds of the various formulas disclosed herein may contain chiral centers, e.g. asymmetric carbon atoms. Thus, the present invention is concerned with the synthesis of both: (i) racemic mixtures of the active compounds, and (ii) enantiomeric forms of the active compounds. The resolution of racemates into enantiomeric forms can be done in accordance with known procedures in the art. For example, the racemate may be converted with an optically active reagent into a diastereomeric pair, and the diastereomeric pair subsequently separated into the enantiomeric forms.

Geometric isomers of double bonds and the like may also be present in the compounds disclosed herein, and all such stable isomers are included within the present invention unless otherwise specified. Also included in active compounds of the invention are tautomers (e.g., tautomers of imidazole) and rotamers.

Active compounds for carrying out methods disclosed herein include compounds of Formula I:

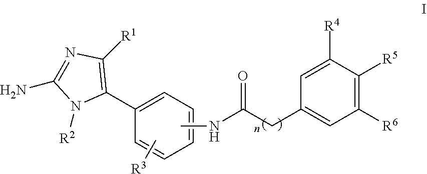

wherein:

R¹, R² and R³ are each independently H or alkyl (e.g., lower alkyl);

n is an integer from 0 to 5; and

R⁴, R³ and R⁶ are each independently H or halo,

or a salt thereof (e.g., a pharmaceutically acceptable salt thereof).

In some embodiments, R⁵ is H; and R⁴ and R⁶ are each independently chloro or bromo.

In some embodiments, the compound of Formula I is a compound of Formula I(a):

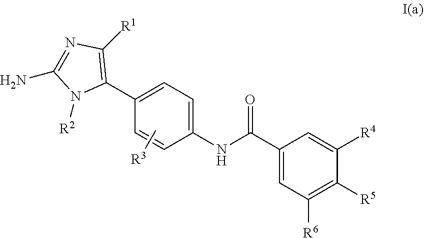

wherein:

R¹, R² and R³ are each independently H or alkyl (e.g., lower alkyl); and

R⁴, R¹ and R⁶ are each independently H or halo,

or a salt thereof (e.g., a pharmaceutically acceptable salt thereof).

Particular examples of compounds of Formula I include, but are not limited to:

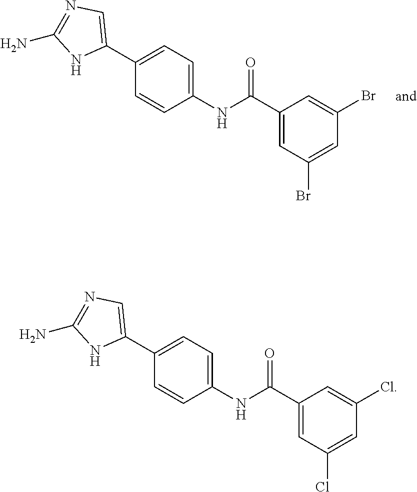 [SMILES: Nc1ncc(-c2ccc(NC(=O)c3cc(Br)cc(Br)c3)cc2)[nH]1 ; "and" ; Nc1ncc(-c2ccc(NC(=O)c3cc(Cl)cc(Cl)c3)cc2)[nH]1]

### C. Methods of Use

As disclosed herein, active compounds can be used to expand the spectrum of activity of Gram-positive antibiotics for use against Gram-negative bacteria. Accordingly, methods for treating a Gram-negative bacterial infection in a subject in need thereof are provided, comprising administering an active compound as taught herein in combination with a Gram-positive antibiotic to said subject in a treatment-effective amount.

“Treating” as used herein refers to any type of activity that imparts a benefit to a patient afflicted with a Gram-negative bacterial infection, including improvement in the condition of the patient (e.g., in one or more symptoms), delay in progression or growth of the infection, etc.

The present disclosure concerns both the treatment of human subjects and animal subjects, particularly mammalian subjects (e.g., mice, rats, dogs, cats, rabbits, and horses), avian subjects (e.g., parrots, geese, quail, pheasant), livestock (e.g., pigs, sheep, goats, cows, chickens, turkey, duck, ostrich, emu), reptile and amphibian subjects, for veterinary purposes or animal husbandry, or for drug screening and development purposes.

“Gram-negative” bacteria are those that do not retain crystal violet dye after an alcohol wash in the Gram staining protocol, due to structural properties in the cell walls of the bacteria. Many genera and species of Gram-negative bacteria are pathogenic. Gram-negative bacteria include members of the phylum proteobacteria, which include, but are not limited to, genus members Acinetobacter, Escherichia, Salmonella, Vibrio, Klebsiella, and Helicobacter. Examples of Gram-negative species include, but are not limited to, Pseudomonas aeuroginosa, Bordetella pertussis, Vibrio vulnificus, Haemophilus influenzae, Halomonas pacifica, Klebsiella pneumoniae, and Acinetobacter baumannii.

A “Gram-positive antibiotic” is an antibiotic used to treat primarily or exclusively Gram-positive bacterial infections, such as a narrow spectrum antibiotic that targets specific types of Gram-positive bacteria. Examples of Gram-positive antibiotics include, but are not limited to, macrolide antibiotics, lipopeptide antibiotics, oxazolidinone antibiotics, and glycopeptide antibiotics.

Macrolide antibiotics include, but are not limited to, azithromycin, clarithromycin, dirithromycin, erythromycin, fidaxomicin, roxithromycin, troleandomycin, telithromycin and spectinomycin.

Lipopeptide antibiotics include, but are not limited to, daptomycin. See also U.S. Pat. No. 6,911,525 to Hill et al.

Oxazolidinone antibiotics include, but are not limited to, eperezolid, linezolid, posizolid, radezolid, ranbezolid, sutezolid, and tedizolid.

Glycopeptide antibiotics include, but are not limited to, vancomycin, teicoplanin, telavancin, ramoplanin and decaplanin.

Also provided are methods to control Gram-negative bacteria, comprising applying an active compound as taught herein in combination with a Gram-positive antibiotic in a Gram-negative bactericidal or bacteristatic effective amount (i.e., amount effective to kill and/or slow the growth of Gram-negative bacteria). “Control” of bacteria as used herein refers to any activity that decreases the amount of bacteria and/or slows the growth thereof. The applying may be carried out, e.g., by applying an active compound and Gram-positive antibiotic to a surface comprising or at risk of comprising Gram-negative bacteria. The applying may be carried out, e.g., by adding the compound and Gram-positive antibiotic to a liquid or material (e.g., gels, foams, fabrics, etc.) comprising or at risk of comprising Gram-negative bacteria.

As used herein, the administration or application of an active compound and antibiotic “in combination” means that they are administered or applied closely enough in time that the administration, application of or presence of one alters the biological effects of the other. They may be administered or applied simultaneously (concurrently) or sequentially.

Simultaneous administration or application may be carried out by mixing the active compound and antibiotic prior to administration or application, or by administering or applying them at the same point in time but at different sites (e.g., anatomic sites) or using different routes of administration or application, or administered or applied at times sufficiently close that the results observed are indistinguishable from those achieved when they are administered or applied at the same point in time.

Sequential administration or application of the active compound and antibiotic may be carried out by administering or applying, e.g., an active compound at some point in time prior to administration or application of an antibiotic, such that the prior administration of active compound enhances the effects of the antibiotic (e.g., increases percentage of bacteria killed and/or slowing the growth of the bacteria). In some embodiments, an active compound is administered or applied at some point in time prior to the initial administration of an antibiotic. Alternatively, the antibiotic may be administered at some point in time prior to the administration or application of an active compound, and optionally, administered or applied again at some point in time after the administration or application of an active compound.

Some aspects of the present invention are described in more detail in the following non-limiting examples.

### EXAMPLES

### Example 1

The 2-aminoimidazole compounds listed in Table 1 were tested for their ability to sensitize the Gram-negative bacterium A. Baumannii to macrolides.

TABLE 1

| Compound | Structure |
|---|---|
| 1 | 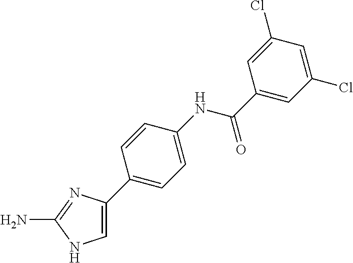 [SMILES: Nc1nc(-c2ccc(NC(=O)c3cc(Cl)cc(Cl)c3)cc2)c[nH]1] |
| 2 | 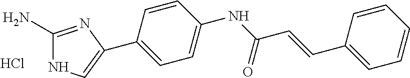 [SMILES: Cl ; Nc1nc(-c2ccc(NC(=O)/C=C/c3ccccc3)cc2)c[nH]1] |
| 3 | 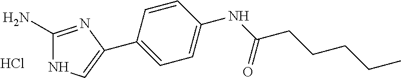 [SMILES: Cl ; CCCCCC(=O)Nc1ccc(-c2c[nH]c(N)n2)cc1] |
| 4 | 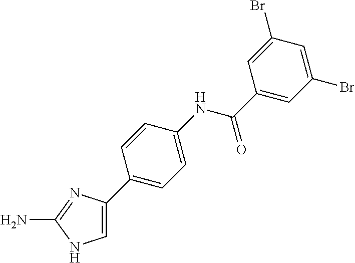 [SMILES: Nc1nc(-c2ccc(NC(=O)c3cc(Br)cc(Br)c3)cc2)c[nH]1] |
| 5 | 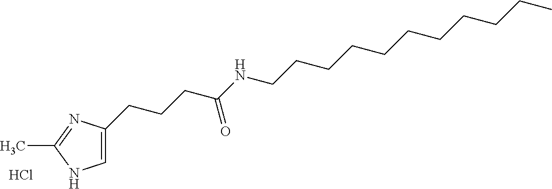 [SMILES: CCCCCCCCCCCNC(=O)CCCc1c[nH]c(C)n1 ; Cl] |
| 6 | 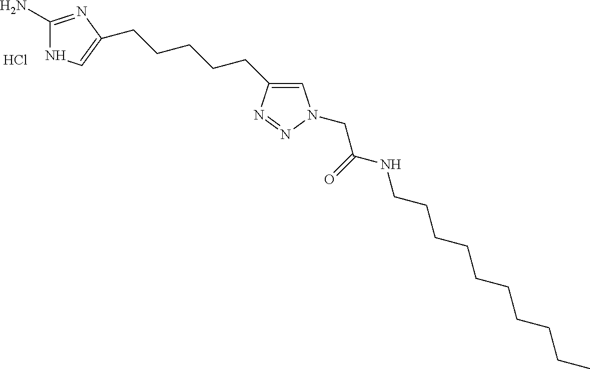 [SMILES: Cl ; CCCCCCCCCCNC(=O)Cn1cc(CCCCCc2c[nH]c(N)n2)nn1] |
| 7 | 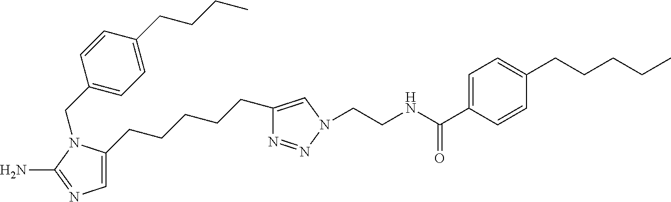 [SMILES: CCCCCc1ccc(C(=O)NCCn2cc(CCCCCc3cnc(N)n3Cc3ccc(CCCC)cc3)nn2)cc1] |
| 8 | 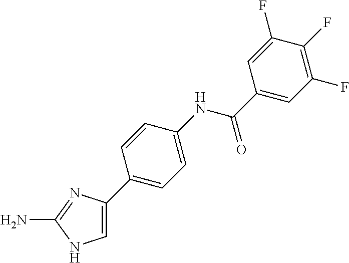 [SMILES: Nc1nc(-c2ccc(NC(=O)c3cc(F)c(F)c(F)c3)cc2)c[nH]1] |
| 9 | 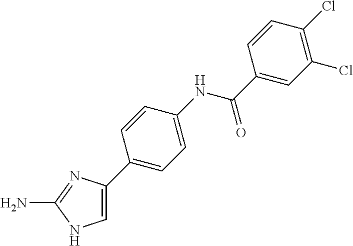 [SMILES: Nc1nc(-c2ccc(NC(=O)c3ccc(Cl)c(Cl)c3)cc2)c[nH]1] |
| 10 | 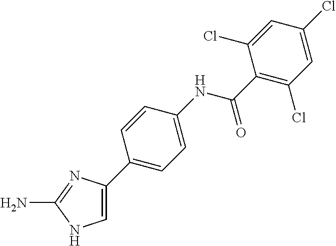 [SMILES: Nc1nc(-c2ccc(NC(=O)c3c(Cl)cc(Cl)cc3Cl)cc2)c[nH]1] |
| 11 | 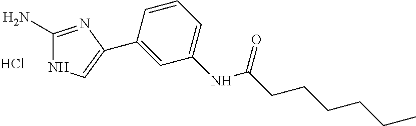 [SMILES: Cl ; CCCCCCC(=O)Nc1cccc(-c2c[nH]c(N)n2)c1] |
| 12 | 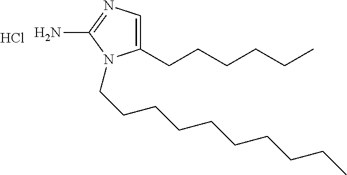 [SMILES: Cl ; CCCCCCCCCCn1c(CCCCCC)cnc1N] |
| 13 | 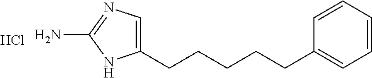 [SMILES: Cl ; Nc1ncc(CCCCCc2ccccc2)[nH]1] |
| 14 | 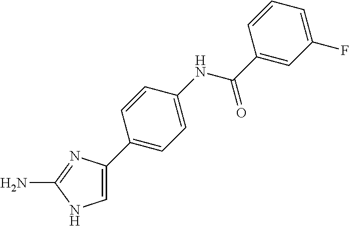 [SMILES: Nc1nc(-c2ccc(NC(=O)c3cccc(F)c3)cc2)c[nH]1] |
| 15 | 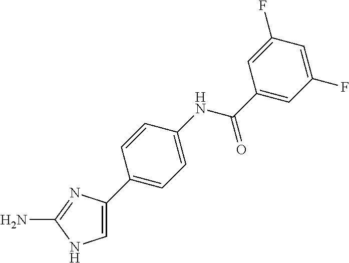 [SMILES: Nc1nc(-c2ccc(NC(=O)c3cc(F)cc(F)c3)cc2)c[nH]1] |
| 16 | 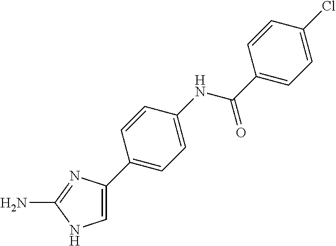 [SMILES: Nc1nc(-c2ccc(NC(=O)c3ccc(Cl)cc3)cc2)c[nH]1] |
| 17 | 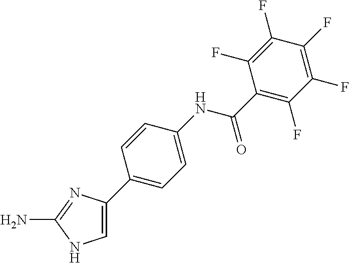 [SMILES: Nc1nc(-c2ccc(NC(=O)c3c(F)c(F)c(F)c(F)c3F)cc2)c[nH]1] |
| 18 | 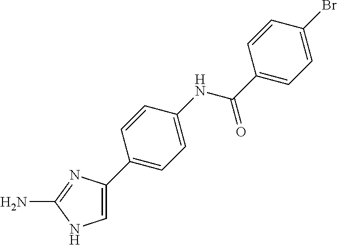 [SMILES: Nc1nc(-c2ccc(NC(=O)c3ccc(Br)cc3)cc2)c[nH]1] |
| 19 | 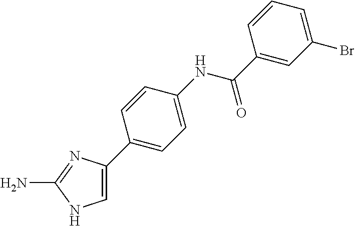 [SMILES: Nc1nc(-c2ccc(NC(=O)c3cccc(Br)c3)cc2)c[nH]1] |
| 20 | 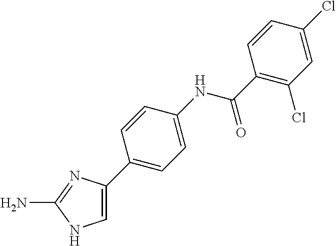 [SMILES: Nc1nc(-c2ccc(NC(=O)c3ccc(Cl)cc3Cl)cc2)c[nH]1] |
| 21 | 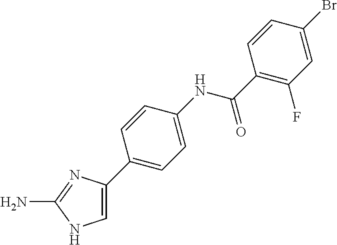 [SMILES: Nc1nc(-c2ccc(NC(=O)c3ccc(Br)cc3F)cc2)c[nH]1] |

Macrolide Sensitization Protocol.

A. baumannii 5075 was plated on LB agar, then grown in CAMHB for 6-8 h and this culture was used to inoculate fresh CAMHB (5×10⁵ CFU/mL). The resulting suspension was aliquoted (5 mL) into culture tubes and compound, from 100 mM DMSO stock, was added. One aliquot was not treated (control). 1 mL of each sample was transferred to a new culture tube and antibiotic was added. Rows 2-12 of a 96-well microtiter plate were filled with 100 μL/well from the remaining 4 mL bacterial subcultures, allowing the concentration of compound to be kept uniform throughout the antibiotic dilution procedure. Samples containing antibiotic were aliquoted (200 μL) into the corresponding first row wells of the microtiter plate. Row 1 wells were mixed 6 to 8 times then 100 μL was withdrawn and transferred to row 2. Row 2 wells were mixed 6 to 8 times, followed by a 100 μL transfer from row 2 to row 3. This procedure was repeated to serially dilute the rest of the rows of the microtiter plate, with the exception of the final row, to which no antibiotic was added (to check for growth of bacteria in the presence of compound alone). The microtiter plate was then covered, placed in a covered damp plastic container and incubated under stationary conditions at 37° C. After 16-18 hrs minimum inhibitory concentration (MIC) values were recorded as the lowest concentration of antibiotic at which there was no visible growth of bacteria. Experiments were carried out in duplicate each time and repeated at least twice. Results are provided in Table 2.

TABLE 2
MIC results from coadministration of compound and macrolide

| | | | Co-administration | | | | | |
|---|---|---|---|---|---|---|---|---|
| | MIC | Concentration | Erythromycin | Fold | Clarithromycin | Fold | Azithromycin | Fold |
| Compound | (μM) | (μM) | MIC (μg/mL) | reduction | MIC (μg/mL) | reduction | MIC (μg/mL) | reduction |
| — | | | 32 | | 32 | | 64 | |
| 1 | 100 | 30 | 4 | 8 | 0.5 | 64 | 8 | 8 |
| 2 | 100 | 30 | 4 | 8 | 1 | 32 | 16 | 4 |
| 3 | >200 | 60 | 2 | 16 | 1 | 32 | 8 | 8 |
| 4 | 100 | 30 | 4 | 8 | 0.25 | 128 | 8 | 8 |
| 5 | 100 | 30 | 2 | 16 | 0.125 | 256 | 8 | 8 |
| 6 | >200 | 60 | 8 | 4 | | | | |
| 7 | 200 | 60 | 8 | 4 | | | | |
| 8 | 100 | 30 | 4 | 8 | 1 | 32 | | |
| 9 | 100 | 30 | 2 | 16 | 0.5 | 64 | | |
| 10 | 100 | 30 | 4 | 8 | 4 | 8 | | |
| 11 | >200 | 60 | 8 | 4 | | | | |
| 12 | 100 | 60 | 8 | 4 | | | | |
| 13 | 200 | 30 | 8 | 4 | | | | |
| 14 | >200 | 60 | 4 | 8 | | | | |
| 15 | 200 | 60 | 4 | 8 | | | | |
| 16 | >200 | 60 | 8 | 4 | | | | |
| 17 | >200 | 60 | 8 | 4 | | | | |
| 18 | >200 | 60 | 8 | 4 | | | | |
| 19 | >200 | 60 | 4 | 8 | | | | |
| 20 | >200 | 60 | 8 | 4 | | | | |
| 21 | >200 | 60 | 4 | 8 | | | | |

The minimum inhibitory concentration (MIC) of each antibiotic was measured in the presence of each compound at a concentration of ≤30% the MIC of the compound. The difference in antibiotic MIC between the control (no compound) sample, and the samples containing each compound was recorded as the fold-reduction.

In general, the compounds exhibited more potent activity toward suppression of clarithromycin resistance compared to either erythromycin or azithromycin, for example compound 1 effects an eight-fold reduction in MIC for erythromycin and azithromycin and a 64-fold reduction in MIC for clarithromycin. Compound 4 exhibited comparable activity with azithromycin and erythromycin, and effected a 128-fold reduction in MIC for clarithromycin.

Surprisingly, the compounds were able to suppress intrinsic resistance of the Gram-negative bacteria to Gram-positive antibiotics, with some showing a reduction in resistance of 64- or 128-fold. This is in contrast to prior work, which demonstrated a resensitization of acquired resistance to an antibiotic that the bacteria had been susceptible to at one time. The mechanism by which this happens is unknown; however it is possible that the compounds are exerting their effects at the cell wall in a way that is not dependent upon membrane permealization or efflux inhibition.

### Example 2

Initially, we screened a diverse set of small molecules selected from our in-house library for their effectiveness to potentiate erythromycin against A. baumannii strain 5075. A. baumannii strain 5075 (AB5075) was selected because of its classification as an ideal model strain due to its high virulence in animal models in comparison to other isolates, multidrug resistance, and availability of the complete transposon library, which will aid in determining the mechanism of action of potential adjuvants. Two screening rounds led to the identification of two lead compounds that were active with macrolides and other Gram-positive selective antibiotics. Initial studies revealed that disruption of LPS synthesis or assembly likely plays a role in the mechanism of action of these adjuvants. Additionally, we showed that this approach is a viable means to overcome intrinsic Gram-negative resistance in vivo using a Galleria mellonella model of A. baumannii infection.

### Results and Discussion

Screening a Variety of 2-Aminoimidazole Compounds for Reduction of Macrolide MICs Against A. baumannii.

Our initial screening began by testing a diverse selection of 2-AI compounds from our internal library for their minimal inhibitory concentrations (MIC) against A. baumannii isolate 5075. Once the MIC of the 2-AI derivatives alone was determined, the MIC of erythromycin, which was used as representative macrolide, in the presence of the 2-AI adjuvants at a concentration of 30% of their MIC was determined. This screen identified three compounds (1-3) that were able to lower the MIC of erythromycin eight-fold or greater as shown in Tables 3A and 3B.

TABLE 3A
Identification of active adjuvants with erythromycin from internal library
screen against AB5075.

| | | Concentration | Erythromycin | Fold |
|---|---|---|---|---|
| Compound | MIC (μM) | tested (μM) | MIC (μg/mL) | Reduction |
| | — | — | 32 | — |
| 1 | 100 | 30 | 4 | 8 |
| 2 | 100 | 30 | 4 | 8 |
| 3 | >200 | 60 | 2 | 16 |

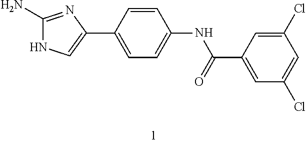 [SMILES: Nc1nc(-c2ccc(NC(=O)c3cc(Cl)cc(Cl)c3)cc2)c[nH]1]

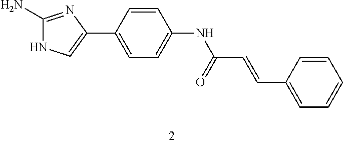 [SMILES: Nc1nc(-c2ccc(NC(=O)/C=C/c3ccccc3)cc2)c[nH]1]

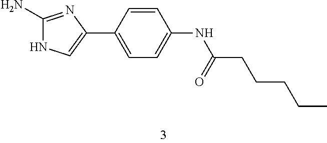 [SMILES: CCCCCC(=O)Nc1ccc(-c2c[nH]c(N)n2)cc1]

TABLE 3B
Initial internal library screen with erythromycin against AB5075.

| Cpd | | | | Erythro- |
|---|---|---|---|---|
| No | | | Concentra- | mycin |
| (if | | MIC | tion | MIC |
| any) | Structure | (μM) | tested (μM) | (μg/mL) |
| — | — | — | — | 32 |
| 1 | 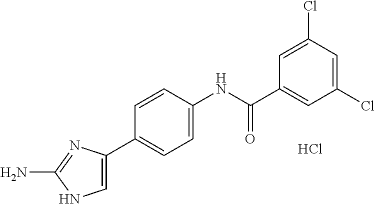 [SMILES: Nc1nc(-c2ccc(NC(=O)c3cc(Cl)cc(Cl)c3)cc2)c[nH]1 ; Cl] | 100 | 30 | 4 |
| 2 | 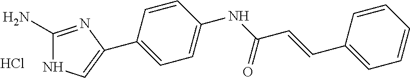 [SMILES: Cl ; Nc1nc(-c2ccc(NC(=O)/C=C/c3ccccc3)cc2)c[nH]1] | 100 | 30 | 4 |
| 3 | 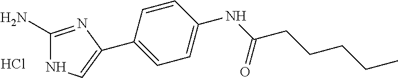 [SMILES: Cl ; CCCCCC(=O)Nc1ccc(-c2c[nH]c(N)n2)cc1] | >200 | 60 | 2 |
| | 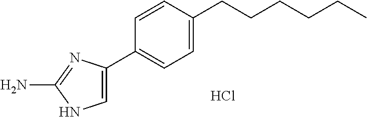 [SMILES: CCCCCCc1ccc(-c2c[nH]c(N)n2)cc1 ; Cl] | 100 | 30 | 16 |
| | 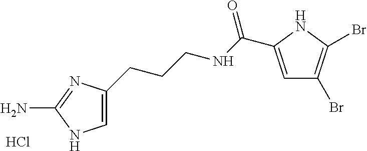 [SMILES: Nc1nc(CCCNC(=O)c2cc(Br)c(Br)[nH]2)c[nH]1 ; Cl] | >200 | 60 | 16 |
| | 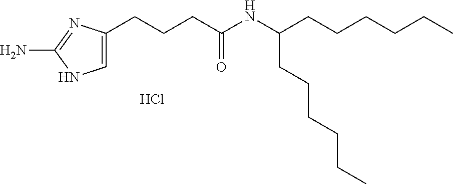 [SMILES: CCCCCCC(CCCCCC)NC(=O)CCCc1c[nH]c(N)n1 ; Cl] | 100 | 30 | 32 |
| | 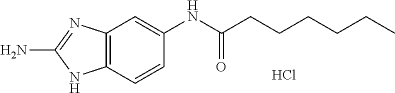 [SMILES: CCCCCCC(=O)Nc1ccc2[nH]c(N)nc2c1 ; Cl] | >200 | 60 | 16 |
| | 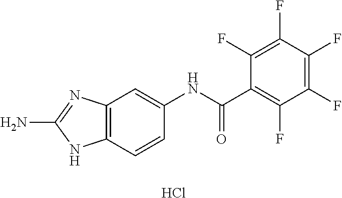 [SMILES: Nc1nc2cc(NC(=O)c3c(F)c(F)c(F)c(F)c3F)ccc2[nH]1 ; Cl] | >200 | 60 | 16 |
| | 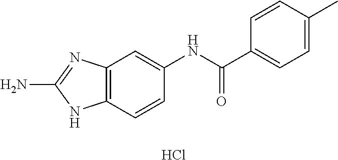 [SMILES: Cc1ccc(C(=O)Nc2ccc3[nH]c(N)nc3c2)cc1 ; Cl] | >200 | 60 | 32 |
| | 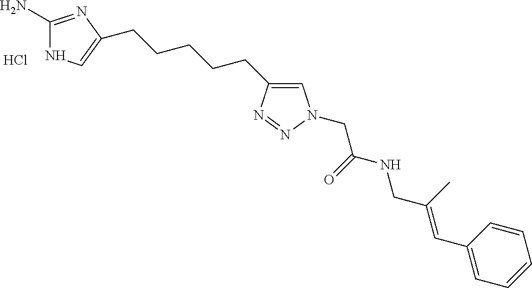 [SMILES: Cl ; C/C(=C\c1ccccc1)CNC(=O)Cn1cc(CCCCCc2c[nH]c(N)n2)nn1] | >200 | 60 | 32 |
| | 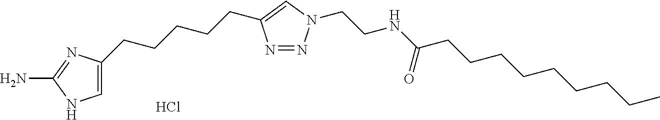 [SMILES: CCCCCCCCCC(=O)NCCn1cc(CCCCCc2c[nH]c(N)n2)nn1 ; Cl] | >200 | 60 | 16 |
| | 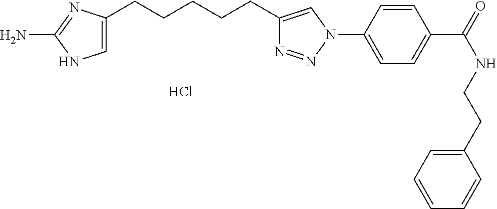 [SMILES: Nc1nc(CCCCCc2cn(-c3ccc(C(=O)NCCc4ccccc4)cc3)nn2)c[nH]1 ; Cl] | >200 | 60 | 32 |
| | 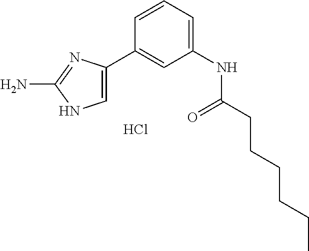 [SMILES: CCCCCCC(=O)Nc1cccc(-c2c[nH]c(N)n2)c1 ; Cl] | >200 | 60 | 8 |
| | 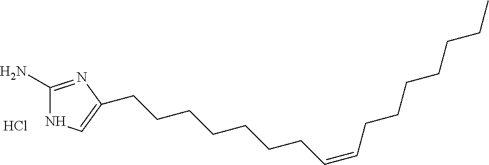 [SMILES: Cl ; CCCCCCC/C=C\CCCCCCCc1c[nH]c(N)n1] | >200 | 60 | 32 |
| | 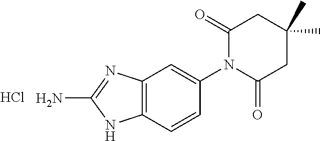 | >200 | 60 | 32 |
| | 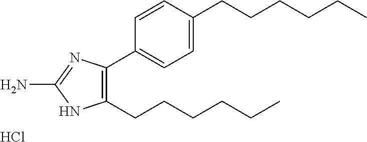 [SMILES: CCCCCCc1ccc(-c2nc(N)[nH]c2CCCCCC)cc1 ; Cl] | >200 | 60 | 32 |
| | 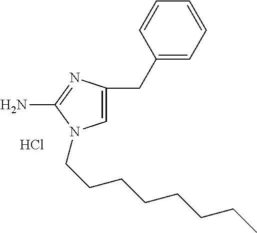 [SMILES: CCCCCCCCn1cc(Cc2ccccc2)nc1N ; Cl] | >200 | 60 | 32 |
| | 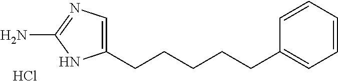 [SMILES: Nc1ncc(CCCCCc2ccccc2)[nH]1 ; Cl] | 200 | 60 | 8 |
| | 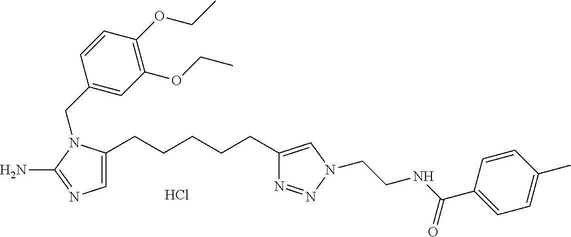 [SMILES: CCOc1ccc(Cn2c(CCCCCc3cn(CCNC(=O)c4ccc(C)cc4)nn3)cnc2N)cc1OCC ; Cl] | 100 | 30 | 32 |

It was noted that all three of compounds 1-3 possessed an aryl 2-AI moiety, so next were screened additional aryl 2-AI compounds for the ability to suppress erythromycin MICs. Previously, it was shown that compound 1 and select derivatives are capable of potentiation of β-lactam antibiotics against A. baumannii and P. aeruginosa. Brackett, C. M., et al., Small-molecule suppression of beta-lactam resistance in multidrug-resistant gram-negative pathogens. J Med Chem, 2014. 57(17): p. 7450-8. The results of this screen are provided in Table 4A.

TABLE 4A
Secondary library screen of aryl 2-AI compounds with erythromycin against
AB5075.

| | | MIC | Concentration | Erythromycin |
|---|---|---|---|---|
| Compound | Structure | (μM) | tested (μM) | MIC (μg/mL) |
| — | — | — | — | 32 |
| 14 | 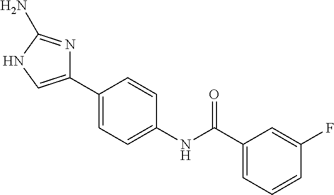 [SMILES: Nc1nc(-c2ccc(NC(=O)c3cccc(F)c3)cc2)c[nH]1] | >200 | 60 | 4 |
| 15 | 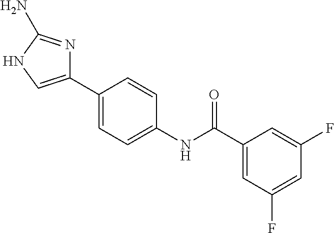 [SMILES: Nc1nc(-c2ccc(NC(=O)c3cc(F)cc(F)c3)cc2)c[nH]1] | 200 | 60 | 4 |
| 10 | 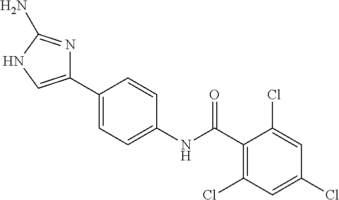 [SMILES: Nc1nc(-c2ccc(NC(=O)c3c(Cl)cc(Cl)cc3Cl)cc2)c[nH]1] | 100 | 30 | 4 |
| 19 | 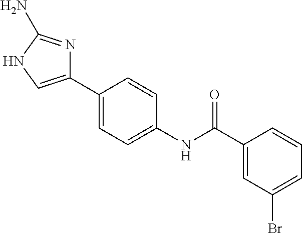 [SMILES: Nc1nc(-c2ccc(NC(=O)c3cccc(Br)c3)cc2)c[nH]1] | >200 | 60 | 4 |
| 8 | 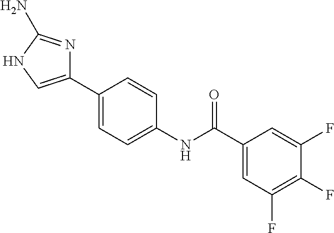 [SMILES: Nc1nc(-c2ccc(NC(=O)c3cc(F)c(F)c(F)c3)cc2)c[nH]1] | 100 | 30 | 4 |
| 9 | 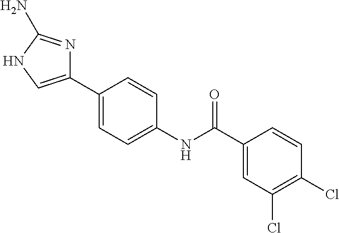 [SMILES: Nc1nc(-c2ccc(NC(=O)c3ccc(Cl)c(Cl)c3)cc2)c[nH]1] | 100 | 30 | 2 |
| 4 | 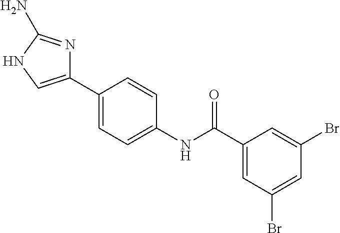 [SMILES: Nc1nc(-c2ccc(NC(=O)c3cc(Br)cc(Br)c3)cc2)c[nH]1] | 100 | 30 | 4 |
| 21 | 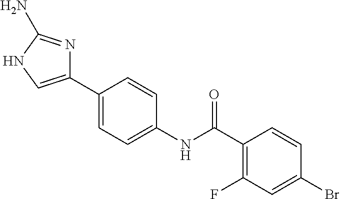 [SMILES: Nc1nc(-c2ccc(NC(=O)c3ccc(Br)cc3F)cc2)c[nH]1] | >200 | 60 | 4 |
| 16 | 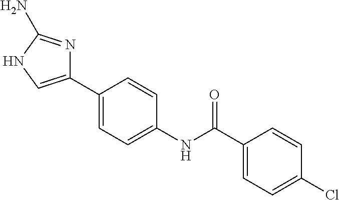 [SMILES: Nc1nc(-c2ccc(NC(=O)c3ccc(Cl)cc3)cc2)c[nH]1] | >200 | 60 | 8 |
| 17 | 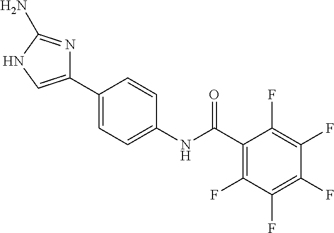 [SMILES: Nc1nc(-c2ccc(NC(=O)c3c(F)c(F)c(F)c(F)c3F)cc2)c[nH]1] | >200 | 60 | 8 |
| 22 | 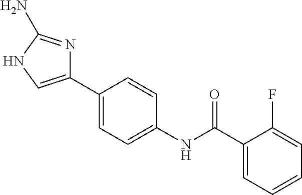 [SMILES: Nc1nc(-c2ccc(NC(=O)c3ccccc3F)cc2)c[nH]1] | >200 | 60 | 16 |
| 18 | 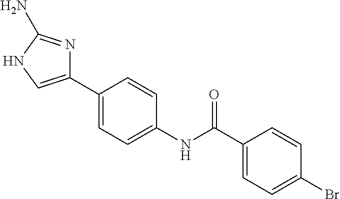 [SMILES: Nc1nc(-c2ccc(NC(=O)c3ccc(Br)cc3)cc2)c[nH]1] | >200 | 60 | 8 |
| 23 | 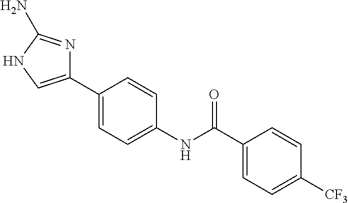 [SMILES: Nc1nc(-c2ccc(NC(=O)c3ccc(C(F)(F)F)cc3)cc2)c[nH]1] | 50 | 15 | 16 |
| 24 | 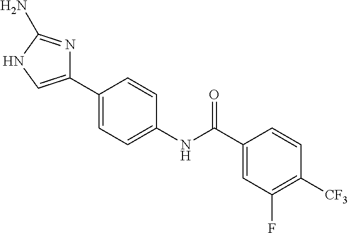 [SMILES: Nc1nc(-c2ccc(NC(=O)c3ccc(C(F)(F)F)c(F)c3)cc2)c[nH]1] | 25 | 7.5 | 16 |
| 25 | 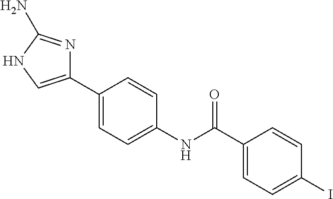 [SMILES: Nc1nc(-c2ccc(NC(=O)c3ccc(I)cc3)cc2)c[nH]1] | >200 | 60 | 32 |
| 20 | 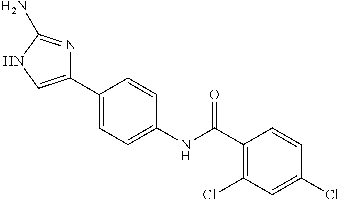 [SMILES: Nc1nc(-c2ccc(NC(=O)c3ccc(Cl)cc3Cl)cc2)c[nH]1] | >200 | 60 | 8 |

As shown in Table 4B, this screen revealed several compounds that sensitized A. baumannii to erythromycin.

TABLE 4B
Identification of active aryl 2-AI compounds from secondary
library screen with erythromycin against AB5075.

| | MIC | Concentration | Erythromycin | Fold |
|---|---|---|---|---|
| Compound | (μM) | tested (μM) | MIC (μg/mL) | Reduction |
| | — | — | 32 | — |
| 14 | >200 | 60 | 4 | 8 |
| 15 | 200 | 60 | 4 | 8 |
| 10 | 100 | 30 | 4 | 8 |
| 19 | >200 | 60 | 4 | 8 |
| 8 | 100 | 30 | 4 | 8 |
| 9 | 100 | 30 | 2 | 16 |
| 4 | 100 | 30 | 4 | 8 |
| 21 | >200 | 60 | 4 | 8 |

To determine the spectrum of activity, several active compounds were next screened for potentiation of an additional macrolide, clarithromycin.

Despite compound 9 being the most active compound for potentiation of erythromycin, it exhibited lower activity than compounds 1 and 4 with clarithromycin. Compounds 10 and 8 showed limited activity with clarithromycin, only lowering the MIC eight- and 32-fold, respectively (Table 5). Since compounds 1 and 4 displayed notable activity with both erythromycin and clarithromycin, they were carried forward for additional analysis of their activity.

TABLE 5
Potentiation of clarithromycin with active
aryl 2-AI compounds against AB5075.

| | Concentration | Clarithromycin | Fold |
|---|---|---|---|
| Compound | Tested (μM) | MIC (μg/mL) | Reduction |
| | — | 32 | — |
| 1 | 30 | 0.25 | 1.28 |
| 10 | 30 | 4 | 8 |
| 8 | 30 | 1 | 32 |
| 9 | 30 | 0.5 | 64 |
| 4 | 30 | 0.25 | 128 |

The additional screening began by testing both compounds for their repotentiation activity with an additional macrolide antibiotic, azithromycin. As depicted in Table 6, both compounds displayed activity comparable to that exhibited with erythromycin, reducing the MIC by eight-fold.

TABLE 6
Potentiation of azithromycin against AB5075
with two most active aryl 2-AI compounds.

| | Concentration | Azithromycin | Fold |
|---|---|---|---|
| Compound | Tested (μM) | MIC (μg/mL) | Reduction |
| | — | 64 | — |
| 1 | 30 | 8 | 8 |
| 4 | 30 | 8 | 8 |

To further probe the range of activity, several macrolide antibiotics, which are either not FDA approved or are commonly used in veterinary clinics, were tested. As seen in Table 7, moderate activity was seen with both compounds and all four antibiotics, troleandomycin, josamycin, spiramycin and oleandomycin. The highest level of activity was observed with josamycin, the MIC of which was reduced 16- and 32-fold in the presence of compound 1 and 4 respectively.

TABLE 7
Screening two most active adjuvants with unusual
macrolide antibiotics against AR5075.

| | Troleando- | Josamycin | Spiramycin | Oleando- |
|---|---|---|---|---|
| | mycin MIC | MIC | MIC | mycin MIC |
| Compound | (μg/mL) | (μg/mL) | (μg/mL) | (μg/mL) |
| | >512 | 128 | >512 | 256 |
| 1 | 32 | 8 | 128 | 64 |
| 4 | 16 | 4 | 64 | 128 |

Growth of A. baumannii in the Presence of Compound 1 Over Time.

To determine the level (if any) of toxicity exhibited by compound 1 alone, a time kill curve was constructed. A. baumannii strain 5075 was grown in the presence of compound 1 at either 20 μM or 30 μM, and samples were plated at 2, 4, 6, 8, and 24 hour time points. Initial toxicity was seen until the 4-hour time point for 20 μM and 8-hour time point for 30 μM; however, growth was similar to the control after 6 hours for 20 μM and 24 hours for 30 μM.

Screening Active Compounds Against Additional Gram-Positive Selective Antibiotics.

Due to the activity displayed with macrolide antibiotics, it was determined whether these compounds could also potentiate additional Gram-positive selective antibiotics. Accordingly, compounds 1 and 4 were screened with antibiotics from the glycopeptide, lipopeptide, and oxazolidinone classes. As displayed in Table 8, notable MIC reductions of 256- and 128-fold with compound 1 and 256- and 32-fold with compound 4 were produced with vancomycin and daptomycin respectively. Potentiation of linezolid was not observed (two- and zero fold reductions).

TABLE 8
Potentiation of additional selective Gram-positive antibiotics
by compounds 1 and 4^{[a]} against AB5075.

| | Vancomycin | Fold | Daptomycm | Fold | Linezolid | Fold |
|---|---|---|---|---|---|---|
| Compound | MIC (μg/mL) | Reduction | MIC (μg/mL) | Reduction | MIC (μg/mL) | Reduction |
| | 256 | — | 2048 | — | 256 | — |
| 1 | 1 | 256 | 16 | 128 | 128 | 2 |
| 4 | 1 | 256 | 64 | 32 | 256 | 0 |

^{[a]}Compounds tested at 30 μM

The original potentiation screenings were performed at 30% of the compound MIC to avoid inherent toxicity. To determine whether the same activity could be achieved at lower concentrations, potentiation assays were also performed at varying compound concentrations. As depicted in Table 9, decreasing the concentration of compounds 1 and 4 from 30 μM to 20 μM decreases the reduction in vancomycin MIC against A. baumannii from 256-fold to 32-fold and 64-fold, respectively. When the concentration of either compound is decreased to 10 μM, there is no potentiation activity observed.

TABLE 9
Dose dependent resistance suppression of vancomycin
in AB5075 using active adjuvants

| | Concentration | Vancomycin |
|---|---|---|
| Compound | Tested (μM) | MIC (μg/mL) |
| | — | 256 |
| 1 | 30 | 1 |
| | 20 | 8 |
| | 15 | 64 |
| | 10 | 256 |
| | 5 | 256 |
| | 4 | 256 |
| 4 | 30 | 1 |
| | 20 | 4 |
| | 15 | 32 |
| | 10 | 128 |
| | 5 | 256 |
| | 4 | 128 |

Unlike the immediate effects of decreasing compound concentration on potentiation of vancomycin, decreasing compound concentration has a lesser effect on the potentiation capabilities of macrolides. As depicted in Table 10 decreasing either compound concentration from 30 μM to 20 μM only reduces the MIC activity by two-fold for all three macrolides. Further decreasing the concentration leads to a gradual decrease in activity. At 5 μM, activity becomes negligible with only two- or four-fold reductions for both compounds with all three macrolide antibiotics.

TABLE 10
Dose dependent resistance suppression of macrolide
antibiotics in AB5075 using active adjuvants

| | | Erythro- | Clarithro- | Azithro- |
|---|---|---|---|---|
| | Concentration | mycin MIC | mycin MIC | mycin MIC |
| Compound | Tested (μM) | (μg/mL) | (μg/mL) | (μg/mL) |
| | — | 32 | 32 | 64 |
| 1 | 30 | 4 | 0.25 | 8 |
| | 20 | 4 | 0.5 | 16 |
| | 15 | 8 | 0.5 | 16 |
| | 10 | 8 | 2 | 16 |
| | 5 | 16 | 16 | 32 |
| | 4 | 16 | 16 | 32 |
| 4 | 30 | 4 | 0.25 | 8 |
| | 20 | 4 | 0.25 | 16 |
| | 15 | 8 | 0.5 | 8 |
| | 10 | 8 | 2 | 32 |
| | 5 | 16 | 8 | 64 |
| | 4 | 16 | 16 | 64 |

As previously stated, A. baumannii 5075 was chosen for these studies due to its virulence and multidrug resistance, but to further analyze the activity of our lead compounds, 23 A. baumannii clinical isolates obtained from Walter Reed Army Institute of Research (WRAIR) were tested. MIC values were determined for clarithromycin and vancomycin against all 23 isolates. A combination of compounds 1 or 4 at 30 μM and clarithromycin or vancomycin were then tested against the 23 isolates. Overall, results indicated that the activity exhibited with the lead compounds and clarithromycin and vancomycin against AB35075 was not unique to strain 5075 and was mostly comparable to the results obtained from the 23 other strains (Table 11)

TABLE 11
Screen for potentiation of clarithromycin and vancomycin using
compounds 1 and 4^{[a]} against A. baumannii isolates.

| | Clarithromycin MIC (μg/mL) | | | Vancomycin MIC (μg/mL) | | |
|---|---|---|---|---|---|---|
| A. baumannii | | With | With | | With | With |
| strain | — | compound 1 | compound 4 | — | compound 1 | compound 4 |
| 3560 | 32 | 0.5 | 0.5 | 256 | 0.5 | 32 |
| 3785 | 64 | 0.5 | 0.5 | 512 | 1 | 128 |
| 3806 | 16 | 0.25 | ≤0.125 | 256 | 2 | 32 |
| 3927 | 32 | 0.5 | 0.5 | 256 | 8 | 64 |
| 4025 | 64 | 0.25 | 0.25 | 512 | 2 | 16 |
| 4026 | 64 | 0.5 | 1 | 512 | 2 | 16 |
| 4027 | 64 | ≤0.25 | 0.25 | 512 | 1 | 32 |
| 4052 | 32 | ≤0.25 | ≤0.25 | >512 | 4 | 64 |
| 4269 | 32 | ≤0.25 | ≤0.25 | 512 | 2 | 64 |
| 4448 | 32 | ≤0.25 | ≤0.25 | 512 | 1 | 4 |
| 4456 | 32 | 0.5 | ≤0.25 | 512 | 16 | 64 |
| 4490 | 32 | 0.5 | ≤0.25 | 256 | 1 | 8 |
| 4498 | 64 | ≤0.25 | ≤0.25 | 512 | 1 | 16 |
| 4795 | 32 | 0.5 | 0.5 | >512 | 4 | 4 |
| 4857 | 64 | 0.5 | 0.5 | — | Toxic | Toxic |
| 4878 | 32 | 0.5 | 1 | 512 | 2 | 1 |
| 4957 | 32 | 0.5 | 0.5 | 512 | 8 | 8 |
| 4991 | 32 | 0.5 | 0.5 | 512 | 32 | 32 |
| 5001 | 32 | 0.5 | 0.5 | 512 | 2 | 1 |
| 5197 | 64 | 0.5 | 0.5 | 512 | 16 | 8 |
| 5256 | 32 | 0.25 | 0.25 | 512 | 2 | 8 |
| 5711 | 64 | 0.5 | 0.5 | >512 | 8 | 64 |
| 8967 | 16 | 0.25 | 0.25 | 512 | 4 | 8 |

^{[a]}Both compounds tested at 30 μM for all strains.

Screening Lead Compounds for Activity with Additional Antibiotics.

As mentioned earlier, these compounds have previously been reported to suppress resistance to β-lactam antibiotics in A. baumannii and other Gram-negative bacteria. We therefore screened the two lead compounds for potentiation of other classes of antibiotics to determine whether they are acting in a non-specific manner. As we observed for linezolid, neither compound showed any notable activity in combination with a range of aminoglycoside antibiotics (Table 12).

TABLE 12
Screening two active compounds with range of aminoglycoside antibiotics against AB5075.

| | Gentamicin | Kanamycin | Tobramycin | Amikacin | Neomycin | Streptomycin |
|---|---|---|---|---|---|---|
| | MIC (μg/mL) | MIC (μg/mL) | MIC (μg/mL) | MIC (μg/mL) | MIC (μg/mL) | MIC (μg/mL) |
| — | >512 | >512 | 256 | 128 | 64 | >512 |
| 1 | >512 | >512 | 64 | 128 | 16 | 256 |
| 4 | >512 | >512 | 64 | 128 | 64 | 512 |

Interestingly however, the presence of the compounds rendered A. baumannii 5075, which is colistin susceptible, considerably more resistant to colistin (Table 13). Compounds 1 and 4 at 30 μM increased the MIC of colistin from 0.5 μg/mL to 16 μg/mL and 4 μg/mL respectively, which according to CLSI breakpoints for A. baumannii represents a shift from susceptible to resistant. Since we saw some toxicity at 30 μM when we analyzed CFU/mL over time, we decided to repeat the repotentiation assays with colistin using only 10 μM of our lead compounds. Despite the decrease in concentration, there was still an increase in MIC with compound 1.

TABLE 13
Effect of the two lead compounds on
susceptibility of AB5075 to colistin.

| | Compound | Colistin | Fold |
|---|---|---|---|
| Compound | Concentration (μM) | MIC (μg/mL) | Increase |
| | | 0.5 | — |
| 1 | 30 | 16 | 32 |
| | 10 | 4 | 8 |
| 4 | 30 | 4 | 8 |
| | 10 | 0.5 | 0 |

Since these results were consistent with an antagonistic relationship between our compounds and colistin, we performed a chequerboard assay to further confirm the relationship. Against AB5075, we determined that the ΣFIC with compound 1 to be 17 and the ΣFIC with compound 10 to be 33. These ΣFIC values indicate that both compounds are highly antagonistic with colistin as a ΣFIC≥2 is considered antagonistic.

In addition to the evaluation of the relationship between compounds 1 and 4 and colistin, the impact of compound 1 on efflux was evaluated using AB5075 and a bisBenzimide H33342 trihydrochloride (H33342) accumulation assay. Using a procedure adapted from Coldham et al. (A 96-well plate fluorescence assay for assessment of cellular permeability and active efflux in Salmonella enterica serovar Typhimurium and Escherichia coli. J Antimicrob Chemother, 2010. 65(8): p. 1655-63) and Richmond et al. (Efflux in Acinetobacter baumannii can be determined by measuring accumulation of H33342 (bis-benzamide). J Antimicrob Chemother, 2013. 68(7): p. 1594-600), the effect of no treatment, a known inhibitor carbonyl cyanide-m-chlorophenyl hydrazone (CCCP), and compound 1 on efflux was analyzed over 30 minutes and in two different treatment variations. Fluorescence was read using excitation and emission filters of 355 and 460 nm, and the values were processed in excel. The first analysis was performed immediately after treating the bacteria with compound 1 at both 10 and 30 μM and CCCP at 25 μM. The results showed only a slight increase in fluorescence in the untreated sample, a notable increase in fluorescence in the sample treated with CCCP, and a decrease in fluorescence for both samples containing compound 1 (Table 14).

TABLE 14
H33342 accumulation analyzed by fluorescence at 355 and 460 nm immediately
following exposure to inhibitor and lead compound 1.

| Time | Untreated | 25 μM CCCP | 10 μM | 30 μM |
|---|---|---|---|---|
| (minutes) | control | inhibitor | compound 1 | compound 1 |
| 0 | 16,338.5 ± 4,353.6 | 42,713.8 ± 10,482.1 | 14,084.3 ± 2,839.3 | 21,152.2 ± 5,161.7 |
| 10 | 16,408.9 ± 4,602.3 | 50,618.3 ± 9,944.3 | 12,812.4 ± 2,718.2 | 21,651 ± 8,731.7 |
| 20 | 17,163.7 ± 4,101.6 | 50,928.8 ± 6,944.8 | 11,627.8 ± 3,119 | 19,748.3 ± 9,182.4 |
| 30 | 22,490.7 ± 3,044.2 | 48,907.2 ± 4,873 | 12,147.8 ± 2,742.8 | 19,562.4 ± 9,574 |

The second analysis was performed after exposing the bacteria to compound 1 at 30 μM for 5-hours shaking at 37° C. Colony counts were preformed after 5-hours to confirm that the compound did not exhibit toxicity. Analysis was performed with H33342 as previously described and the results revealed comparable fluorescence values in both the untreated and compound 1 treated samples (Table 15). This data reveals that neither short nor long term exposure to compound 1 inhibits efflux of AB5075.

TABLE 15
H33342 accumulation analyzed by fluorescence at 355 and
460 nm following exposure to lead compound 1 for 5 hours.

| Time | Untreated | 30 μM |
|---|---|---|
| (minutes) | control | compound 1 |
| 0 | 12,311.3 ± 2,203.1 | 12,226.7 ± 4,671.7 |
| 10 | 16,049 ± 3,924.2 | 15,247 ± 5,499.3 |
| 20 | 27,719.2 ± 3,966.5 | 25,603.1 ± 5,212.9 |
| 30 | 39,768.9 ± 1,052.8 | 33,441.1 ± 3,260.3 |

Since efflux did not seem to be inhibited by compound 1, a backlight assay was performed to determine the lead compounds effect on membrane permeability. The assay was initially performed at 10, 20 and 30 μM with compounds 1 and 4 (Table 16). This assay revealed that at 30 μM compounds 1 and 4 disrupt approximately 83 and 87% of the cellular membrane respectively. When the concentration is lowered to 20 μM, the percent of membrane disrupted also decreases to 78% and 83% respectively. When further decreasing the concentration to 10 μM, the membrane disruption decreases to 39 and 44% respectively. Due to the similar structure and lack of potentiation activity, compound 25 was screened at 10, 20 and 30 μM to compare membrane permeability with that of active compounds. The percent disruption was less than seen with compounds 1 and 4 being 53, 54 and 60% respectively. This data correlates increased permeability to increased activity in A. baumannii 5075.

TABLE 16
Backlight assay to determine membrane permeability
of AB 5075 after exposure to lead compounds.

| | Concentration | Disrupted bacterial |
|---|---|---|
| Compound | Tested (μM) | membrane (%) |
| 1 | 30 | 82.8 |
| | 20 | 78 |
| | 10 | 38.8 |
| 4 | 30 | 87.4 |
| | 20 | 83.2 |
| | 10 | 44 |
| 25 | 30 | 60 |
| | 20 | 54 |
| | 10 | 53.7 |

The effect of the compounds on colistin susceptibility provides insight into their mode of action. It has been shown that inactivation of lipid A production, or downregulation of genes essential for LPS production in A. baumannii correlates to an increase in colistin resistance. The fact that these compounds increase the colistin MIC and do not inhibit efflux suggests that their mechanism of action may involve modulation of LPS production or composition.

Screening of Mutants Identified by Tn-Seq.

In an effort to determine the mode of action, Tn-seq was used to identify and isolate a small library of A. baumannii transposon mutants, which were more sensitive to vancomycin, clarithromycin and compound 1. During individual MIC screening, two mutants, AB522 and AB1310, were identified due to their increased sensitivity to the three macrolide antibiotics and vancomycin when compared to AB 5075 (Table 17).

TABLE 17
MIC values of four main antibiotics against
A. baumannii mutants isolated from Tn-seq.

| | MIC (μg/mL) | | | |
|---|---|---|---|---|
| Strain | Vancomycin | Erythromycin | Clarithromycin | Azithromycin |
| 522 | 64 | 2 | 2 | 4 |
| 1310 | 64 | 4 | 2 | 4 |

Compounds 1 and 4 were screened against both mutants to determine their MIC values (Table 18) and potentiation activity of vancomycin, erythromycin, clarithromycin, and azithromycin.

TABLE 18
MIC values of lead compounds against A. baumannii
mutants isolated from Tn-seq.

| | Compound 1 | Compound 4 |
|---|---|---|
| Strain | MIC (μM) | (μM) |
| 522 | 12.5 | 12.5 |
| 1310 | 12.5 | 12.5 |

The MIC values for both compounds alone against both mutant strains were 12.5 μM, which is notably lower than the MIC values observed with AB5075. When performing the potentiation assays, the first concentration tested was 3 μM, which is slightly less than 30% of the compounds MIC, to avoid inherent toxicity. There was no notable reduction in antibiotic MIC observed with either compound and both strains 522 and 1310. Due to the lack of activity observed at 3 μM, a dose response assay was performed with the highest concentration 40% of the compounds' MIC (Tables 19 and 20). Even at the highest concentration, no potentiation was observed. These results vastly contrast the results observed with AB5075, which could indicate the gene mutated in AB522 and AB1310 is targeted by our lead compounds 1 and 4.

TABLE 19
Dose dependent resistance suppression of vancomycin, erythromycin,
clarithromycin and azithromycin in AB522 using lead compounds.

| | Conc. | Vancomycin | Erythromycin | Clarithromycin | Azithromycin |
|---|---|---|---|---|---|
| Compound | Tested | MIC (μg/mL) | MIC (μg/mL) | MIC (μg/mL) | MIC (μg/mL) |
| | | 64 | 2 | 2 | 4 |
| 1 | 2 | 64 | 2 | 1 | 4 |
| | 3 | 64 | 4 | 1 | 8 |
| | 4 | 32 | 4 | 1 | 4 |
| | 5 | 32 | 2 | 0.5 | 4 |
| | 2 | 64 | 2 | 1 | 8 |
| | 3 | 64 | 2 | 1 | 4 |
| | 4 | 32 | 2 | 1 | 4 |
| | 5 | 32 | 2 | 0.5 | 4 |

TABLE 20
Dose dependent resistance suppression of vancomycin, erythromycin,
clarithromycin and azithromycin in AB1310 using lead compounds.

| | Conc. | Vancomycin | Erythromycin | Clarithromycin | Azithromycin |
|---|---|---|---|---|---|
| Compound | Tested | MIC (μg/mL) | MIC (μg/mL) | MIC (μg/mL) | MIC (μg/mL) |
| | | 64 | 4 | 2 | 4 |
| 1 | 2 | 64 | 4 | 1 | 4 |
| | 3 | 64 | 2 | 1 | 4 |
| | 4 | 64 | 4 | 0.5 | 4 |
| | 5 | 32 | 2 | 0.5 | 4 |
| 4 | 2 | 64 | 4 | 2 | 4 |
| | 3 | 32 | 2 | 1 | 4 |
| | 4 | 32 | 2 | 1 | 4 |
| | 5 | 32 | 2 | 0.5 | 4 |

In Vivo Studies with G. mellonella Model.

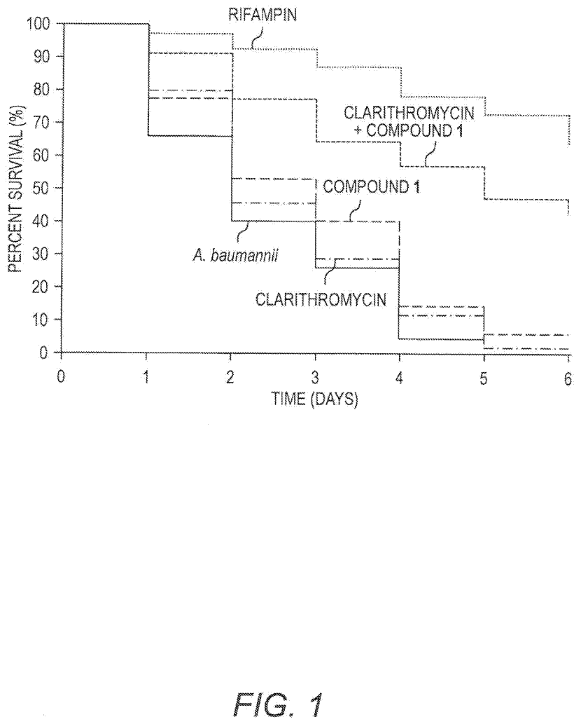

Finally, we tested the in vivo application of compound 1 in combination with clarithromycin in a G. mellonella model infected with AB5075. In this model, G. mellonella worms were infected with AB5075 and either received no treatment, treatment with clarithromycin alone, treatment with compound 1 alone, or a combination treatment of clarithromycin and compound 1 (FIG. 1). Treatment with rifampin at 30 mg/kg was used as a negative control and led to a 64% survival after 6 days. The presence of no treatment or treatment with clarithromycin alone at 25 mg/kg revealed a 1% survival after 6 days. When the worms were treated with compound 1 alone at 100 mg/kg, minimal improvement to 5% survival was noted. Despite the low percent survival seen after treatment with compound 1 alone and clarithromycin alone, the combination treatment revealed a 42% survival rate. While these results are modest, it is important to note that that percent survival was increased after one treatment with the combination of clarithromycin and compound 1. Furthermore, as stated previously A. baumannii strain 5075 is more virulent than most A. baumannii strains, which creates a higher threshold for evaluating activity.

Conclusion.

In conclusion, an initial screen identified several active compounds for their effectiveness to potentiate erythromycin against A. baumannii strain 5075. A secondary screen led to the identification of two active compounds, 1 and 4. Compounds 1 and 4 were capable of lowering the MIC of erythromycin eight-fold, clarithromycin 128-fold, and azithromycin eight-fold. Additional Gram-positive antibiotics were screened to identify the range of activity provided by the lead compounds. A. baumannii strain 5075 was sensitized to vancomycin and daptomycin, with MICs reduced by 256- and 128/32-fold respectively. No activity was seen in combination with either linezolid or a range of aminoglycoside antibiotics, but both compounds increased the MIC of colistin 32- and eight-fold rendering A. baumannii 5075 notably more resistant. The increased colistin resistance and absence of efflux pump inhibition in the presence of the compounds suggests the mode of action has to do with alteration in LPS production or composition. Additional mode of action studies will be reported in due time. In addition to the in vitro studies, in vivo studies were performed using a G. mellonella model. The in vivo studies confirmed that in the presence of clarithromycin or compound 1 alone the percent survival is minimal, but treatment with clarithromycin and compound 1 together increases percent survival from 1% to 42%.

Bacterial Strains, Media, and Antibiotics.

A. baumannii clinical isolate 5075 was obtained from Dr. Colin Manoil at The University of Washington. Colonies were grown on solid LB agar. Cation-adjusted Mueller-Hinton Broth (CAMHB) (catalog number 212322) was purchased from BD Diagnostics. Vancomycin (catalog number V2002) was purchased from Sigma-Aldrich. Erythromycin (catalog number 45673) was purchased from Fluka. Clarithromycin (catalog number C2220) and azithromycin (catalog number A2076) were purchased from TCL. Daptomycin (catalog number RD002) was purchased from TSZ Chemical. All assays were completed in duplicate and were repeated at least two separate times. All compounds were dissolved as their HCl salts in molecular biology grade DMSO as 100 mM stock solutions. Vancomycin was dissolved in sterile water while erythromycin, clarithromycin, azithromycin and daptomycin were dissolved in molecular biology grade DMSO.

Broth Microdilution Method for MIC Determination of A. baumannii.

Day Cultures (6 h) were subcultured to 5×10⁵ CFU/mL in cation adjusted Mueller-Hinton broth (CAMHB). Aliquots (1 mL) were placed in culture tubes, and compound was added from 100 mM stock solutions, such that the compound concentration equaled the highest concentration tested (200 μM or 64 μg/mL). Samples were then aliquoted (200 μL) into the first row of wells of a 96-well plate, with all remaining wells being filled with 100 μL of initial bacterial subculture. The wells in row one were mixed five times before 100 μL was transferred to the following row (row two). Row two was then mixed five times, and 100 μL was transferred to row three. This processed was repeated until the final row had been mixed. This process provided a serial dilution of the compound. Plates were covered with Glad Press n'Seal and were incubated under stationary conditions at 37° C. for 16 hours. MIC values were recorded as the lowest concentration at which no bacterial growth was observed.

Broth Microdilution Method for Antibiotic Resensitization of A. baumannii.

Day Cultures (6 h) were subcultured to 5×10⁵ CFU/mL in cation adjusted Mueller-Hinton broth (CAMHB). Aliquots (4 mL) were placed in culture tubes, and compound was added from 100 mM stock solutions, such that the compound concentration was 30% of the MIC of the compound against the particular bacterial strain. One milliliter of the resulting solution was aliquoted into a separate culture tube and was dosed with antibiotic so that the resulting concentration was the highest desirable concentration to be tested. Bacteria treated with antibiotic alone were used as a control. Row one of a 96-well plate was filled with 200 μL of the antibiotic/compound solution, and the remaining rows were filled with 100 μL of the remaining 4 mL bacterial subculture. The wells in row one were mixed five times before 100 μL was transferred to the following row (row two). Row two was then mixed five times, and 100 μL was transferred to row three. This process was repeated until the second to last row had been reached. The last row is left unexposed to antibiotic in order to serve as a negative control. The antibiotic only treated bacteria was plated by aliquoting 200 μL of the treated bacteria into row one and filling the remaining rows with untreated bacteria from the original bacteria subculture. The rows were mixed in the same way as described above. Plates were covered with Glad Press n'Seal and were incubated under stationary conditions at 37° C. for 16 hours. MIC values were recorded as the lowest concentration at which no bacterial growth was observed. Fold reductions were determined by comparison of the compound treated wells with the antibiotic only treated control wells.

Time Kill Curves.

A. baumannii 5075 was grown overnight (16 hours) in CAMHB at 37° C. with shaking. This culture was then subcultured to 5×10⁵ CFU/mL in fresh CAMHB. The inoculated media was aliquoted (3 mL) into culture tubes, and the compound was added from 100 mM stock solutions. Untreated inoculated culture tubes served as the control, and tubes were incubated at 37° C. with shaking. Samples were taken at 2, 4, 6, 8, and 24 hour time points and were serially diluted in fresh CAMHB and plated on LB agar. Plates were incubated under stationary conditions overnight at 37° C. After incubation, colonies were enumerated and growth curves were constructed.

Checkerboard Assay for FIC Determination.

Day Cultures (6 h) of A. baumannii 5075 were subcultured to 5×10⁵ CFU/mL in cation adjusted Mueller-Hinton broth (CAMHB). 100 μL of the inoculated media was distributed into all wells of a 96-well plate except well 1A. Inoculated CAMHB (200 μL) containing compound (twice the highest concentration tested) was added to well 1A, and 100 μL of the same sample was added to wells 2A-12A. The contents in column A were mixed five times before 100 μL was transferred to column B. The contents of column B were mixed 5 times before transferring to column C. This process was repeated with the remainder of the columns up to column G. Column H was not mixed to allow determination of the antibiotic MIC alone. Inoculated media (100 μL) containing colistin at twice the highest concentration tested was added to wells A1-H1 and was serially diluted in the same way as described above. Row 12 was not mixed so that the MIC of the compound alone could be determined. The MICs of both compound and antibiotic alone were recorded, as well as the wells containing the combination. The ΣFICs were calculated as follows: ΣFIC=FIC (Compound)+FIC (antibiotic), where FIC (compound) is the MIC of the compound in the combination/MIC of the compound alone and the FIC (antibiotic) is the MIC of the antibiotic in combination/the MIC of the antibiotic alone. The combination is considered synergistic when the ΣFIC is ≤0.5, additive between 0.5 and 2, and antagonistic when the ΣFIC≥2.

In Vivo Analysis Using Galleria mellonella.

Galleria mellonella larvae (Speedy Worm, Alexandria, Minn.) were used within ten days of shipment from the vendor. After reception of worms, larvae were kept in the dark at room temperature for at least 24 h before infection. Larvae weighing between 200 to 300 mg were used in the survival assay. Using a 10 μL glass syringe (Hamilton, Reno, Nev.) fitted with a 30 G needle (Exel International, St. Petersburg, Fl), 5 μL of the desired compound and concentration were injected into the last left proleg. After 2.5 h, a second 5 μL injection containing 6×10⁵ CFU of Acinetobacter baumannii 5075 was injected into the second to last left proleg. Injected worms were left at room temperature in the dark while being assessed at 24 h intervals over 6 days. Larvae were considered dead if they did not respond to physical stimuli. Experiment was repeated 7 times using 10 larvae per experimental group.

The foregoing is illustrative of the present invention, and is not to be construed as limiting thereof. The invention is defined by the following claims, with equivalents of the claimed to be included therein.

1. A method to treat a Gram-negative bacterial infection in a subject in need thereof, comprising administering a compound of Formula I: 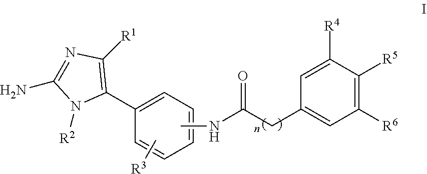
wherein:
R¹, R² and R³ are each independently H or alkyl;
n is an integer from 0 to 5; and
R⁴, R⁵ and R⁶ are each independently H or halo,
or a pharmaceutically acceptable salt thereof,
in combination with a Gram-positive antibiotic to said subject in a treatment-effective amount.

2. The method of claim 1, wherein said Gram-positive antibiotic is a macrolide antibiotic, a lipopeptide antibiotic, an oxazolidinone antibiotic, or a glycopeptide antibiotic.

3. The method of claim 1, wherein said Gram-positive antibiotic is a macrolide antibiotic.

4. The method of claim 1, wherein said Gram-positive antibiotic is erythromycin.

5. The method of claim 1, wherein said Gram-positive antibiotic is clarithromycin.

6. The method of claim 1, wherein said Gram-positive antibiotic is azithromycin.

7. The method of claim 1, wherein said Gram-negative bacterial infection comprises bacteria of the genus Acinetobacter, Escherichia, Salmonella, Vibrio, Klebsiella, and/or Helicobacter.

8. The method of claim 1, wherein said Gram-negative bacterial infection comprises bacteria of the genus Acinetobacter.

9. The method of claim 1, wherein said Gram-negative bacterial infection comprises bacteria of the species Pseudomonas aeuroginosa, Bordetella pertussis, Vibrio vulnifcus, Haemophilus influenzae, Halomonas pacifica, Klebsiella pneumoniae, and/or Acinetobacter baumannii.

10. The method of claim 1, wherein said Gram-negative bacterial infection comprises bacteria of the species Acinetobacter baumannii.

11. The method of claim 1, wherein R⁵ is H; and R⁴ and R⁶ are each independently chloro or bromo.

12. The method of claim 1, wherein said compound of Formula I is a compound of Formula I(a): 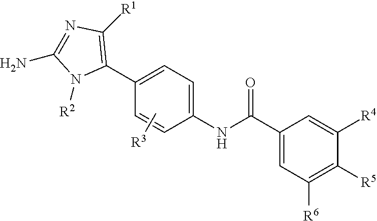
wherein:
R¹, R² and R³ are each independently H or alkyl; and
R⁴, R⁵ and R⁶ are each independently H or halo,
or a pharmaceutically acceptable salt thereof.

13. The method of claim 1, wherein said compound of Formula I is: 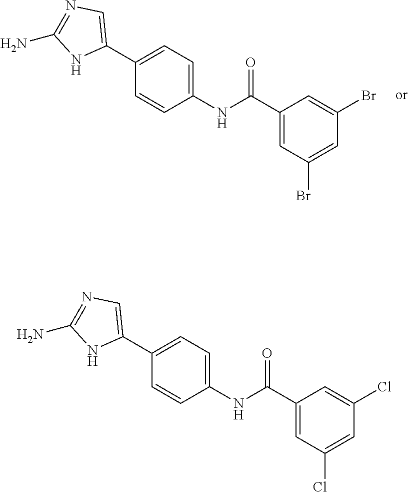 [SMILES: Nc1ncc(-c2ccc(NC(=O)c3cc(Br)cc(Br)c3)cc2)[nH]1 ; "or" ; Nc1ncc(-c2ccc(NC(=O)c3cc(Cl)cc(Cl)c3)cc2)[nH]1]
or a pharmaceutically acceptable salt thereof.

14. A method to control Gram-negative bacteria, comprising applying a compound of Formula I: 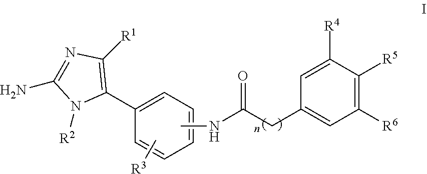
wherein:
R¹, R² and R³ are each independently H or alkyl;
n is an integer from 0 to 5; and
R⁴, R⁵ and R⁶ are each independently H or halo,
or a salt thereof,
in combination with a Gram-positive antibiotic in a Gram-negative bactericidal-effective amount.

15. The method of claim 14, wherein said applying is carried out by applying the compound and Gram-positive antibiotic to a surface comprising or at risk of comprising said Gram-negative bacteria.

16. The method of claim 14, wherein said applying is carried out by adding the compound and Gram-positive antibiotic to a liquid or material (e.g., gels, foams, fabrics, etc.) comprising or at risk of comprising said Gram-negative bacteria.

17. The method of claim 14, wherein said Gram-positive antibiotic is a macrolide antibiotic, a lipopeptide antibiotic, an oxazolidinone antibiotic, or a glycopeptide antibiotic.

18. The method of claim 14, wherein said Gram-negative bacteria comprises bacteria of the genus Acinetobacter, Escherichia, Salmonella, Vibrio, Klebsiella and/or Helicobacter.

19. The method of claim 14, wherein R⁵ is H; and R⁴ and R⁶ are each independently chloro or bromo.

20. The method of claim 14, wherein said compound of Formula I is a compound of Formula I(a): 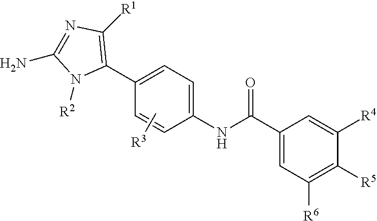
wherein:
R¹, R² and R³ are each independently H or alkyl; and
R⁴, R⁵ and R⁶ are each independently H or halo,
or a salt thereof.

21. The method of claim 14, wherein said compound of Formula I is: 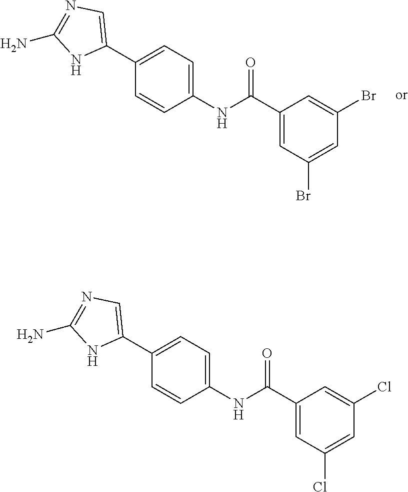 [SMILES: Nc1ncc(-c2ccc(NC(=O)c3cc(Br)cc(Br)c3)cc2)[nH]1 ; "or" ; Nc1ncc(-c2ccc(NC(=O)c3cc(Cl)cc(Cl)c3)cc2)[nH]1]
or a salt thereof.
